(19) United States
(12) Patent Application Publication
(10) Pub. No.: US 2017/0369551 A1
(43) Pub. Date: Dec. 28, 2017

(54) METHODS OF TREATING IgA NEPHROPATHY AND HENOCH-SCHONLEIN PURPURA NEPHRITIS USING A B-CELL ACTIVATING FACTOR (BAFF) INHIBITOR

(71) Applicant: ANTHERA PHARMACEUTICALS, INC., Hayward, CA (US)

(72) Inventors: Renee MARTIN, Los Altos Hills, CA (US); James PENNINGTON, Lafayette, CA (US); Colin HISLOP, Menlo Park, CA (US); Charles OLSON, Moraga, CA (US)

(21) Appl. No.: 15/632,257

(22) Filed: Jun. 23, 2017

Related U.S. Application Data
(60) Provisional application No. 62/354,576, filed on Jun. 24, 2016.

(51) Int. Cl. C07K 14/705; A61K 38/17; A61K 45/06

(52) U.S. Cl. CPC C07K 14/70575; A61K 45/06; A61K 38/177; C07K 2319/74; C07K 2319/31

(57) ABSTRACT

Provided herein are methods, compositions, and kits for treating IgA nephropathy and Henoch-Schönlein purpura nephritis using BAFF inhibitors, including blisibimod.

### PRIORITY CLAIM

This application claims priority to U.S. Provisional Application No. 62/354,576, filed Jun. 24, 2016, which is incorporated herein by reference in its entirety, including drawings.

### BACKGROUND

Immunoglobulin A nephropathy (IgAN), the most common form of primary glomerulonephritis in the world, is characterized by deposition of immune complexes of IgA in the glomeruli of the kidneys. IgAN is associated with progressive kidney damage and loss of renal function, culminating in end stage renal disease requiring dialysis or renal transplantation in approximately 50% of patients over 20 years (Koyama 1997). Longitudinal analyses show that elevated proteinuria is prognostic of risk of progression to end-stage renal disease, and remission of proteinuria to <1 g/24 hr substantially improves prognosis (Reich 2007; Coppo 2015).

The pathology of IgAN is thought to involve increased circulating levels of underglycosylated IgA1 and antibodies directed against this immunogenic species. Together these form IgA-containing immune complexes that accumulate in the glomerular mesangium, provoking mesangial proliferation and inflammation (Suzuki 2011).

B cell activating factor (BAFF) is a mediator of B cell differentiation, maturation, and survival. Its importance in the treatment of systemic lupus erythematosus (SLE) is under intense investigation. Elevated expression of BAFF has been reported in IgA nephropathy, and this elevated expression correlates with greater disease activity and higher histological score (Xin 2013).

Although treatment of SLE with BAFF inhibitors is known in the art, no previous studies have evaluated or demonstrated a therapeutic benefit of BAFF inhibition in subjects with IgA nephropathy or Henoch-Schönlein purpura nephritis.

### SUMMARY

Provided herein in certain embodiments are methods of treating IgA nephropathy in a subject in need thereof comprising administering a therapeutically effective amount of a BAFF inhibitor. In certain embodiments, the BAFF inhibitor is blisibimod, and in certain of these embodiments blisibimod is administered in its monomeric form, its dimeric form, or as a mixture of monomeric and dimeric forms. In certain embodiments, the BAFF inhibitor is administered in combination with one or more second therapeutic agents. Also provided herein are compositions and kits for use in these methods.

Provided herein in certain embodiments are methods of treating Henoch-Schönlein purpura nephritis in a subject in need thereof comprising administering a therapeutically effective amount of a BAFF inhibitor. In certain embodiments, the BAFF inhibitor is blisibimod, and in certain of these embodiments blisibimod is administered in its monomeric form, its dimeric form, or as a mixture of monomeric and dimeric forms. In certain embodiments, the BAFF inhibitor is administered in combination with one or more second therapeutic agents. Also provided herein are compositions and kits for use in these methods.

### BRIEF DESCRIPTION OF DRAWINGS

FIG. 1: Effects of blisibimod on urinary protein:creatinine ratio in subjects with SLE.

FIG. 2: Effects of blisibimod on autoantibodies, peripheral B cells, C3, C4 and serum immunoglobulins in subjects with SLE.

FIG. 3: Proposed mechanism by which BAFF promotes disease pathology in IgA nephropathy.

FIG. 4: Proposed mechanism by which blisibimod rescues disease pathology in IgA nephropathy.

FIG. 5: Exemplary dimeric blisibimod N-terminal variants.

FIG. 6: Effects of blisibimod on urinary protein:creatinine ratio when administered in addition to an angiotensin-converting enzyme (ACE) inhibitor or angiotensin II receptor blocker (ARB) in subjects with IgA nephropathy.

FIG. 7: Effects of blisibimod on peripheral B cells and immunoglobulins when administered in addition to an ACE inhibitor or ARB in subjects with IgA nephropathy.

### DETAILED DESCRIPTION

In subjects with SLE, disease activity is known to correlate with both BAFF expression and the titer of anti-double-stranded DNA autoantibodies (Petri 2008). Treatment with BAFF inhibitors such as blisibimod has been shown to improve SLE disease activity, with concomitant decreases in the number of peripheral B cells, plasma cells (from which the majority of immunoglobulins are secreted), immunoglobulins (including IgG, IgM and IgA), and autoantibodies.

IgA nephropathy is diagnosed by positive staining for IgA against a spectrum of typical histological changes in renal biopsy tissue. Disease activity and histological severity in renal tissue from IgA nephropathy patients have been shown to correlate with BAFF expression (Xin 2013). Further, transgenic mice that overexpress BAFF exhibit elevated IgA expression and develop nephrotic disease when exposed to an infectious agent (McCarthy 2011). However, it has never been established that BAFF plays a pathologic role in IgA nephropathy, and no side-by-side evaluation of SLE and IgA nephropathy has been performed previously to evaluate whether the diseases arise from similar aberrations in the immune system.

SLE and IgA nephropathy differ substantially with respect to clinical presentation and course of disease, populations affected, characteristics of pathogenic immune complexes including size and composition (targeting immunoglobulin subclasses and their molecular targets), site of immune complex deposition within the kidney (highly dependent upon physical characteristics of the immune complexes, including size, charge, and Fc receptors), renal histopathology, and response to various treatments.

Clinical manifestations of IgAN are typically restricted to the kidney (hematuria, proteinuria, decreased GFR), whereas SLE is a protean disease affecting multiple organ systems including the kidney. IgAN affects males and females equally and is more prevalent in western Asia, whereas SLE is primarily a female disease with a stronger racial than geographic predilection. The clinical course of IgAN is typically insidious, with progression over decades, whereas the course of SLE is typically more acute, with periods of activity and remission. The immune complexes found in IgAN are comprised of underglycosylated IgA1 and targeting IgG, IgM, and/or IgA antibodies; underglycosylated IgA1 is produced primarily in mucosal lymphoid tissue, perhaps in response to infection or other antigenic challenge, and is inherently immunogenic. Immune complexes found in SLE are typically composed of IgG or IgM targeting various nuclear antigens. Immune complexes are found in the glomerular mesangium in IgAN, whereas the immune complexes in lupus nephritis (inflammation of the kidney occurring in some patients with SLE) are typically localized to the glomerular subendothelium or subepithelium, with subsequent differences in histopathology both between the two diseases and within the spectrum of histological and clinical patterns found in lupus nephritis.

Treatment strategies for SLE have been generally unsuccessful in IgA nephropathy. Unlike in SLE, where corticosteroids or immunosuppressants are the mainstays of therapy (Bertsias 2012), treatment of IgA nephropathy is generally limited to controlling the renin-angiotensin system using angiotensin converting enzymes and angiotensin receptor blockers. Addition of immunosuppressants or corticosteroids to this treatment regimen provide no clear incremental benefit (Rauen 2015). While some studies report improvements in proteinuria and serum creatinine or eGFR following treatment with corticosteroids (Lv 2009; Pozzi 1999; Pozzi 2004), other studies of adults and children have reported that addition of corticosteroids to ACEI-reduced proteinuria did not affect renal function after long-term treatment (Katafuchi 2003; Hogg 2006). The clearest steroid effect was reported from the recent NEFIGAN trial in which a significant reduction in proteinuria (p=0.0066) was observed following nine months of therapy with oral budesonide, a gastrically restricted corticosteroid that targets Peyer's patches at the ileocecal junction (“Pharmalink Announces Positive Result in Phase 2b Trial of Nefecon®,” Apr. 14, 2015, http://www.prnewswire.com/news-releases/pharmalink-announces-positive-result-in-phase-2b-trial-of-nefecon-499654311.html). However, emerging data in prospective, randomized, controlled trials ends the debate over the potential benefit of systemic corticosteroids. Specifically, no improvement in efficacy was observed with corticosteroid therapy on top of supportive ACEI/ARB therapy versus with supportive therapy alone in the STOP-IgAN trial (NCT00554502), which followed 162 subjects with biopsy-proven IgAN for three years (Floege 2016a). Further, the evaluation of the long-term efficacy and safety of oral methylprednisolone on a background of routine ACEI/ARB therapy in the TESTING study (NCT01560052) was recently halted due to a safety signal in the corticosteroid arm (Floege 2016b).

Based on the above differences, one of ordinary skill in the art would not expect treatment modalities that work for SLE to work for IgA nephropathy.

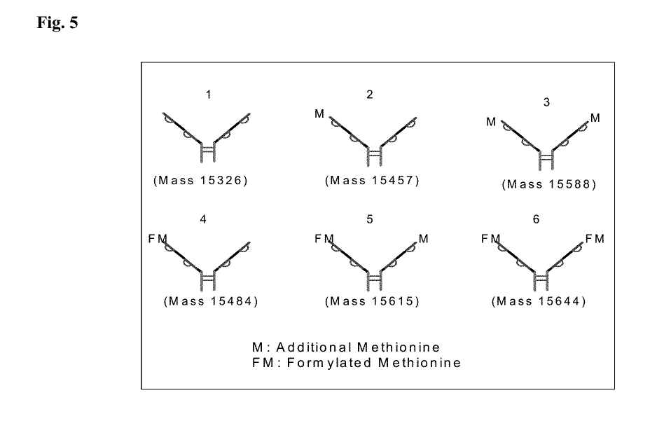

Blisibimod (CAS Registry No. 1236126-45-6) is a potent, selective inhibitor of soluble and membrane-bound BAFF. Blisibimod is a 291 amino acid peptibody having the amino acid sequence: MGCKWDLLIKQWVCDPLGSGSATGGSGSTASSGSGSATHMLPGCKWDLLIKQWVCDP LGGGGGVDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKALP APIEKTISKAKGQPREPQVYTLPPSRDELTKNQVSLTCLVKGFYPSDIAVEWESNGQPEN NYKTTPPVLDSDGSFFLYSKLTVDKSRWQQGNVFSCSVMHEALHNHYTQKSLSLSPGK (SEQ ID NO:1). Residues 2-17 and 41-58 are BAFF-binding peptides, residues 18-40 and 59-64 are linkers, and residues 65-291 are an IgG1 Fc domain. The N-terminal methionine residue may be cleaved off, for example by methionine aminopeptidases, following administration. Alternatively, the N-terminal methionine may be removed prior to administration, or left off entirely. When present, the N-terminal methionine may be in the form of a modified form of methionine such as des-methionyl, methionyl, or formyl methionyl. Blisibimod can form a homodimer in which the two substituent polypeptides are held together via disulfide bridges. In such homodimers, the N-terminal methionine may be present or absent from both monomeric substituents. Alternatively, blisibimod may form a heterodimer in which the N-terminal methionine or a modified form thereof is present on one subunit but not the other, or in which the N-terminal methionine is a modified form of methionine on one subunit and a non-modified form or a different modified form of methionine on the other. Exemplary blisibimod homodimers and heterodimers are set forth in FIG. 5. Blisibimod may be administered in either its monomeric or dimeric form.

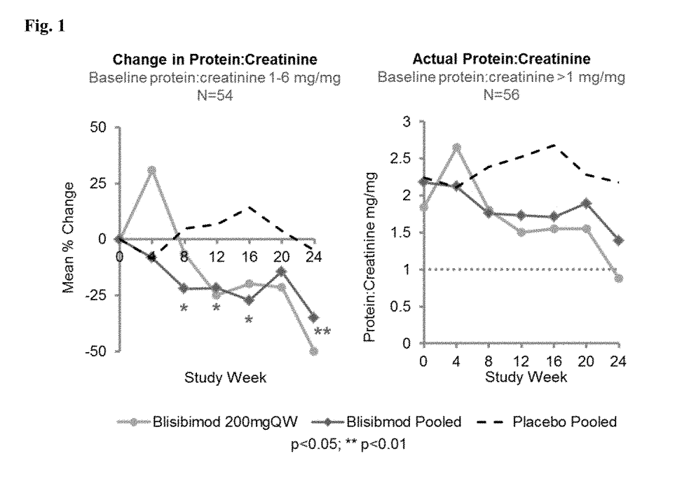

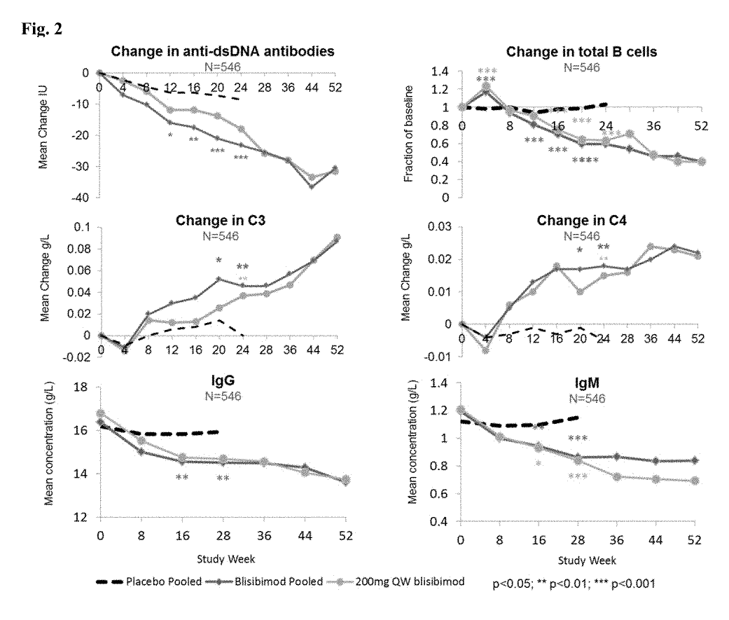

Four clinical trials have been completed with blisibimod in subjects with SLE, and one in subjects with B-cell chronic lymphocytic leukemia. One of these studies, the Phase 2b PEARL-SC study (clinical trial identifier NCT01162681) evaluated the efficacy and safety of blisibimod in 547 subjects with seropositive SLE. Subjects were randomized 3:1:1:1 to receive placebo or blisibimod at one of three dosages: 100 mg/week (QW), 200 mg QW, or 200 mg Q4W for 24 to 52 weeks (Furie 2015). A significant improvement in the proportion of subjects achieving the SLE Responder Index was observed with blisibimod (200 mg QW) versus placebo in the subgroup of subjects with high disease activity at enrollment (Furie 2015). Furthermore, in a subgroup of subjects with baseline urinary protein:creatinine ratios of 1-6 mg/mg, treatment with blisibimod resulted in a significantly greater reduction in protein:creatinine ratio than placebo from weeks 8 through 24 (−50.1% [n=21] versus −5.1% [n=22] at week 24) (FIG. 1) (Furie 2015). Concomitant and significant reductions in serum concentrations of anti-dsDNA autoantibodies and immunoglobulins IgG and IgM were also observed in subjects receiving blisibimod, as well as significant increases in serum complement C3 and C4 (FIG. 2) (Furie 2015; Furie 2013).

The phase 2 BRIGHT-SC study (clinical trial identifier NCT02062684) enrolled 58 subjects with biopsy-proven IgA nephropathy. The primary objective of the study was to compare the effects of blisibimod and placebo on urinary protein:creatinine ratio. The study was originally designed as a phase 2/3 study targeting an enrollment of 200 subjects, but due to slow recruitment randomization was curtailed after 1.5 years and the study was converted to phase 2. The primary endpoint was week 24, but subjects could elect to continue blinded treatment for up to two years.

Subjects in BRIGHT-SC were randomized to receive blisibimod or placebo via subcutaneous (SC) injection as follows:
Blisibimod: 100 mg administered 3 times/week for 8 weeks, followed by 200 mg once/week; or
Placebo: injections matched in volume to blisibimod, administered 3 times/week for 8 weeks, followed by once/week.

As disclosed herein, interim futility and efficacy analyses performed over the course of the BRIGHT-SC study have shown that blisibimod administration results in favorable effects relative to placebo with regard to several critical markers of disease activity in subjects with IgA nephropathy. Specifically, IgA nephropathy patients receiving blisibimod exhibited favorable trends in proteinuria as measured by urinary protein:creatinine ratio, and also exhibited decreased levels of peripheral B cells and serum immunoglobulins (IgA, IgG, and IgM). The pattern of proteinuric response observed in the IgAN population differed from that observed in the SLE population, which is not surprising given the different pathogeneses of the two diseases and the baseline level of proteinuria in the studies.

Overall, the BRIGHT-SC study data suggest that BAFF inhibition, by reducing peripheral B cells, activated B cells, and immunoglobulins, may prevent deterioration of urinary protein:creatinine levels (Barratt 2016), and may improve renal function over time in IgAN. Since these effects were observed in the presence of standard-of-care ACE inhibitor or ARB treatment, they suggest that BAFF inhibition may present a new therapeutic modality for patients with the disease.

Based on the findings set forth herein, methods are provided for treating IgA nephropathy in a subject in need thereof by administering a therapeutically effective amount of a BAFF inhibitor. In certain of these embodiments, the BAFF inhibitor is blisibimod. Also provided herein are compositions and kits for use in these methods.

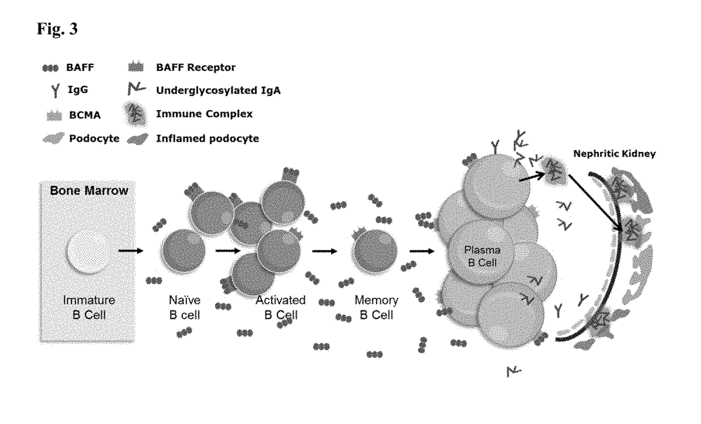

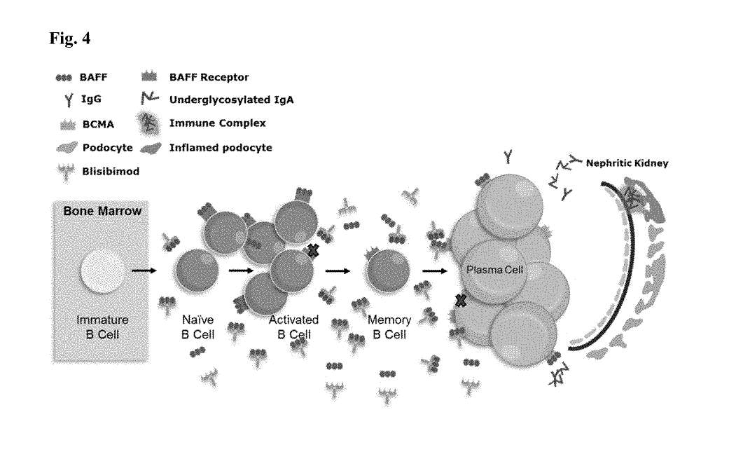

Without wishing to be bound by any theory, a mechanism is proposed whereby, similar to the relationship between anti-dsDNA autoantibodies and renal manifestations in SLE (Linnik 2005), renal damage in IgA nephropathy is attributed to immune complexes that contain autoreactive antibodies. Under such a mechanism, a BAFF inhibitor such as blisibimod may be used to rescue disease activity in IgA nephropathy by decreasing B cell maturation and plasma cell genesis, thereby inhibiting secretion of both pathogenic, poorly glycosylated IgA1 and the autoreactive immunoglobulins that target the IgA1 underglycosylated hinge region. This results in a decrease in the formation of pathogenic immune complexes that activate mesangial cells, recruit inflammatory infiltrates, damage renal tissue, and increase urinary protein loss, with a consequent reduction in glomerular and interstitial inflammation (FIG. 3 and FIG. 4). Additionally, a more general immunosuppressive effect of blisibimod is proposed herein based on its known ability to decrease the number of peripheral B cells (Furie 2015).

A common pathology with overlapping clinical presentations has been previously established between IgA nephropathy and Henoch-Schönlein purpura nephritis across multiple studies and countries. The evidence for this common pathology includes similarities in morphological and immunohistochemical manifestations characterized by renal biopsy evidence of increased IgA-containing immune complexes, mesangial cell proliferation, and IgA deposits at the mesangial area in glomeruli (Wyatt 2013; Davin 2001; Oh 2012). In addition, subjects in both disease groups progress toward end-stage renal disease, with slower progression in IgA nephropathy. The key differences between the diseases lie in the age of onset (typically younger Henoch-Schönlein purpura nephritis; Komatsu 2016), severity of clinical manifestations (typically higher in Henoch-Schönlein purpura nephritis; Komatsu 2016), and the presence of certain systemic manifestations in Henoch-Schönlein purpura nephritis such as skin purpura (which gives the disease its name), arthritic joints, and gastrointestinal symptoms (e.g., abdominal pain, fecal blood, nausea, vomiting, constipation, or diarrhea). In addition, Henoch-Schönlein purpura nephritis presents within one month after the appearance of anaphylactoid purpura in 80% of cases (Davin 2001; Oh 2012; Yoshikawa 1987; Waldo 1988). However, in the remaining ˜20% of cases, renal symptoms precede the purpura, suggesting that the underlying cause may be similar to IgA nephropathy. This is further supported by a Japanese study showing that 53 subjects with IgA nephropathy, six (11%) developed the skin manifestations of Henoch-Schönlein purpura (Kamei 2016). This led the authors to suggest that IgA nephropathy and Henoch-Schönlein purpura nephritis are different manifestations of and should be considered variants of a single disease (Kamei 2016).

Accordingly, one of ordinary skill in the art would expect that the beneficial effects observed in subjects with IgA nephropathy following treatment with a BAFF inhibitor such as blisibimod would predict benefit to subjects with Henoch-Schönlein purpura nephritis. Thus, further provided herein are methods for treating Henoch-Schönlein purpura nephritis in a subject in need thereof by administering a therapeutically effective amount of a BAFF inhibitor. In certain of these embodiments, the BAFF inhibitor is blisibimod. Also provided herein are compositions and kits for use in these methods.

A “BAFF inhibitor” as used herein refers to any molecule that inhibits the activity of BAFF either partially or completely, and either selectively or non-selectively. For example, a BAFF inhibitor may inhibit BAFF activity by blocking BAFF protein activity or binding, decreasing the stability of BAFF protein, or decreasing BAFF expression at the mRNA or protein level. In certain embodiments, a BAFF inhibitor for use in the methods, compositions, and kits provided herein is blisibimod. In other embodiments, the BAFF inhibitor may be another biologic, including for example another peptibody or an antibody or polypeptide (e.g., soluble or membrane-bound BAFF), a small molecule, or a nanoparticle.

The terms “treat,” “treating,” and “treatment” as used herein with regard to a disease such as IgA nephropathy or Henoch-Schönlein purpura nephritis refers to one or more of preventing the development of the disease, slowing the onset or rate of development of the disease, reducing the risk of developing the disease, preventing or delaying the development of symptoms associated with the disease, reducing or ending symptoms associated with the disease, altering the level of one or more biomarkers associated with the disease, completely or partially curing the disease, or combinations thereof. For example, in certain non-limiting embodiments, “treating” IgA nephropathy may refer to slowing or eliminating progressive loss of renal function, and or generating an acute or chronic reduction in urinary protein levels.

A “subject in need thereof” as used herein refers to a mammalian subject, preferably a human, who has been diagnosed with IgA nephropathy or Henoch-Schönlein purpura nephritis, is suspected of having IgA nephropathy or Henoch-Schönlein purpura nephritis, and/or exhibits one or more symptoms associated with IgA nephropathy or Henoch-Schönlein purpura nephritis. In certain embodiments, the subject may have previously received one or more therapeutic interventions for the treatment of IgA nephropathy or Henoch-Schönlein purpura nephritis.

A “therapeutically effective amount” of a BAFF inhibitor or a second therapeutic agent as used herein is an amount of the BAFF inhibitor or second therapeutic agent that produces a desired therapeutic effect in a subject, such as treating IgA nephropathy or Henoch-Schönlein purpura nephritis. In certain embodiments, the therapeutically effective amount is an amount predicted to yield the most effective results in terms of therapeutic efficacy in a given subject. The precise therapeutically effective amount of a BAFF inhibitor or second therapeutic agent may vary depending on a variety of factors, including but not limited to the characteristics of the BAFF inhibitor or second therapeutic agent (e.g., activity, pharmacokinetics, pharmacodynamics, bioavailability), the physiological condition of the subject (e.g., age, body weight, body surface area, sex, disease type and stage, renal function, volume of distribution, medical history, general physical condition, responsiveness to a given dosage, other present medications), the nature of any additional pharmaceutically acceptable agents in a composition comprising the BAFF inhibitor and/or second therapeutic agent, and the route of administration. As an example, a therapeutically effective amount of a BAFF inhibitor for administration to a pediatric subject may be significantly lower than a therapeutically effective amount for an adult with similar disease characteristics. A therapeutically effective amount may be adjusted over time through routine experimentation, for example by monitoring a subject's response to administration of the BAFF inhibitor or second therapeutic agent and adjusting the dosage accordingly.

In certain embodiments, the methods provided herein utilize a composition comprising a BAFF inhibitor. Accordingly, in certain embodiments methods are provided for treating IgA nephropathy or Henoch-Schönlein purpura nephritis in a subject in need thereof comprising administering to the subject a composition comprising a therapeutically effective amount of a BAFF inhibitor. Further provided herein are compositions for use in these methods, i.e., compositions comprising a therapeutically effective amount of a BAFF inhibitor, as well as kits comprising such compositions. In certain embodiments, a composition comprising a BAFF inhibitor further comprises one or more pharmaceutically acceptable excipients, including for example one or more carriers, diluents, disintegrants, binders, fillers, bulking agents, lubricants, glidants, buffering agents, or preservatives. For example, in certain embodiments wherein the BAFF inhibitor is blisibimod, the composition may comprise one or more of sodium acetate, sucrose, mannitol, dextrose, glycerol, sorbitol, histidine, and/or polysorbate 80. In certain embodiments, a composition comprising a BAFF inhibitor may comprise one or more additional pharmaceutically acceptable agents, including for example one or more colorants or flavoring agents.

In certain embodiments of the methods provided herein, the BAFF inhibitor is administered by injection, for example by intraperitoneal, intraarterial, intracapsular, intracardiac, intradermal, intramuscular, intrapulmonary, intraspinal, intrasternal, intrathecal, intrauterine, intravenous, subarachnoid, subcapsular, subcutaneous, transmucosal, or transtracheal injection. In other embodiments, the BAFF inhibitor is administered by another route, including for example oral, pulmonary, nasal, or transdermal administration. In those embodiments wherein the BAFF inhibitor is administered orally, the BAFF inhibitor may be formulated as a composition in a solid dosage form, including but not limited to a tablet, capsule, pill, troche, lozenge, cachet, or pellet. In other embodiments, the BAFF inhibitor may be formulated as a composition for liposomal or proteinoid encapsulation.

In certain embodiments of the methods provided herein wherein the BAFF inhibitor is blisibimod, blisibimod may be administered as a monomer, a dimer (e.g., a homodimer), or a mixture of monomers and dimers. For example, in certain embodiments the methods provided herein comprise administering a therapeutically effective amount of monomeric blisibimod, dimeric blisibimod (e.g., a homodimer), or a combination thereof. In certain embodiments wherein a combination of monomeric and dimeric blisibimod are administered, the ratio of monomeric to dimeric blisibimod may be known, calculated, or estimated prior to administration. In certain embodiments, the methods provided herein utilize blisibimod with the N-terminal methionine residue (residue 1 in SEQ ID NO:1), blisibimod without the N-terminal methionine residue, or a mixture of blisibimod with the N-terminal methionine residue and blisibimod without the N-terminal methionine residue. Such mixtures may include, for example, a mixture of monomers with and monomers without the N-terminal methionine; a mixture of homodimers in which both substituents include the N-terminal methionine and homodimers in which the N-terminal methionine is absent on both substituents; or heterodimers in which one substituent includes the N-terminal methionine and the other does not. In those embodiments wherein the N-terminal methionine residue is present, the methionine may be unmodified, or it may be a modified form of methionine such as des-methionyl, methionyl, or formyl methionyl. In certain embodiments wherein the N-terminal methionine residue is present at the time of administration, the residue may be cleaved off all or some of the administered blisibimod molecules following administration. In certain embodiments, a composition comprising blisibimod for use in the methods provided herein comprises a mixture of monomeric and dimeric forms of blisibimod and/or a mixture of blisibimod molecules with or without an intact N-terminal methionine (wherein the N-terminal methionines are unmodified, modified, or a mixture thereof). Exemplary blisibimod dimers are set forth in FIG. 5. In certain embodiments of the methods provided herein, the IgG1 Fc domain of blisibimod (residues 65-291 of SEQ ID NO:1) may comprise one or more modifications, including for example one or more substitutions, deletions, or insertions. Such substitutions or insertions may comprise naturally occurring or non-naturally occurring amino acids.

In certain embodiments of the methods provided herein, the BAFF inhibitor is administered over a treatment period determined in advance. For example, the BAFF inhibitor may be administered over a total treatment period of 1 week, 2 weeks, 1 month, 2 months, 6 months, 1 year, or more than 1 year. In other embodiments, the BAFF inhibitor is administered over a variable treatment period, e.g., until a specific therapeutic benchmark is reached. In certain embodiments, the BAFF inhibitor is administered at a fixed dosage over the entire treatment period. In other embodiments, the BAFF inhibitor is administered at different dosages over the course of the treatment period. For example, the dosage may be increased or decreased over time based on therapeutic effect, patient tolerance, or other factors. In certain embodiments, the BAFF inhibitor is administered at a higher induction dosage at the start of the treatment period, then lowered to a maintenance dosage for the remainder of the treatment period.

In certain embodiments of the methods provided herein wherein the BAFF inhibitor is blisibimod, blisibimod is administered at a dosage of 50 mg or more per administration (e.g., 50 mg, 75 mg, 100 mg, 150 mg, 200 mg, 250 mg, or 300 mg per administration). In the BRIGHT-SC study, the dosage of blisibimod (200 mg/300 mg) was limited in part by pragmatic considerations such as desired injection volume and the desire to limit the number of injections. In certain situation, higher dosages may become feasible and/or desirable. Thus, in certain embodiments blisibimod may be administered at a dosage greater than 300 mg per administration (e.g., 350 mg, 400 mg, 450 mg, 500 mg, or more than 500 mg per administration). In certain embodiments, the dosage of blisibimod may be calculated based on a subject's body weight. For example, in certain embodiments blisibimod may be administered at a dosage of 0.3 mg/kg or more per administration (e.g., 0.3 mg/kg, 1 mg/kg, 2 mg/kg, 6 mg/kg, 10 mg/kg, or more than 10 mg/kg per administration).

In certain embodiments of the methods provided herein, a BAFF inhibitor is administered in combination with one or more second therapeutic agents. Accordingly, provided herein are methods of treating IgA nephropathy or Henoch-Schönlein purpura nephritis in a subject in need thereof comprising administering to said subject a BAFF inhibitor and one or more second therapeutic agents. Examples of suitable second therapeutic agents include, but are not limited to, ACE inhibitors such as benazepril (Lotensin®), captopril, enalapril (Vasotec®), fosinopril, lisinopril (Zestril®), moexipril (Univasc®), perindopril (Aceon®), quinapril (Accupril®), Ramipril (Altace®), or trandolapril (Mavik®); ARBs such as azilsartan (Edarbi®), candesartan (Atacand®), eprosartan (Teveten®), irbesartan (Avapro®), losartan (Cozaar®), olmesartan (Benicar®), telmisartan (Micardis®), or valsartan (Diovan®); B-cell targeted therapeutic agents such as rituximab (Rituxan®) or belimumab (Benlysta®); and corticosteroids such as betamethasone, dexamethasone, fluocortolone, methylprednisolone, paramethasone, prednisolone, prednisone, triamcinolone, hydrocortisone, cortisone, prednylidene, rimexolone, deflazacort, cloprednol, meprednisone, or cortivazol.

In certain embodiments of the methods provided herein that comprise administering a BAFF inhibitor and one or more second therapeutic agents, the BAFF inhibitor and the one or more second therapeutic agents may be administered simultaneously. In certain of these embodiments, the BAFF inhibitor and one or more second therapeutic agents may be administered as part of a single composition. Accordingly, provided herein in certain embodiments are compositions comprising a BAFF inhibitor and one or more second therapeutic agents, as well as kits comprising such compositions and methods of using such compositions to treat IgA nephropathy or Henoch-Schönlein purpura nephritis in a subject in need thereof. In other embodiments wherein the BAFF inhibitor and the one or more second therapeutic agents are administered simultaneously, they are administered via separate compositions. Accordingly, provided herein in certain embodiments are methods of treating IgA nephropathy or Henoch-Schönlein purpura nephritis in a subject in need thereof comprising simultaneously administering a first composition comprising a BAFF inhibitor and a second composition comprising one or more second therapeutic agents. In these embodiments, the first and second compositions may be administered by the same or different routes.

In certain embodiments of the methods provided herein that comprise administering a BAFF inhibitor and one or more second therapeutic agents, the BAFF inhibitor and one or more second therapeutic agents may be administered sequentially via separate compositions. Accordingly, provided herein in certain embodiments are methods of treating IgA nephropathy or Henoch-Schönlein purpura nephritis in a subject in need thereof comprising sequentially administering a first composition comprising a BAFF inhibitor and a second composition comprising one or more second therapeutic agents. In these embodiments, the first and second compositions may be administered by the same or different routes. The BAFF inhibitor and second therapeutic agent may be administered at the same interval (e.g., 3 times/week, 2 times/week, 1 time/week, biweekly, or 1 time/month) or at different intervals. In certain embodiments, the BAFF inhibitor and one or more second therapeutic agents may be administered over the same treatment period (e.g., both agents may be administered for 1 week, 2 weeks, 1 month, 2 months, 6 months, 1 year, or more than 1 year). In other embodiments, the BAFF inhibitor and one or more second therapeutic agents may be administered over different treatment periods, such that one is administered over a longer time course than the other. In certain embodiments, a subject may have been treated with the one or more second therapeutic agents prior to receiving the first administration of the BAFF inhibitor. In certain of these embodiments, administration of the one or more second therapeutic agents continues after the first BAFF inhibitor administration. In other embodiments, treatment with the one or more second therapeutic agents is permanently or temporarily discontinued after the first BAFF inhibitor administration. In those embodiments wherein two or more second therapeutic agents are administered to the subject, the two or more second therapeutic agents may be administered via a single composition or separate compositions, by the same or different routes, at the same or different intervals, and over the same or different treatment periods.

In certain embodiments of the methods provided herein, a combination of two or more different BAFF inhibitors may be administered to the subject. Accordingly, provided herein in certain embodiments are methods of treating IgA nephropathy or Henoch-Schönlein purpura nephritis in a subject in need thereof comprising administering to said subject two or more BAFF inhibitors, alone or in combination with one or more second therapeutic agents. Also provided herein are compositions comprising two or more BAFF inhibitors, as well as kits comprising such compositions and methods of using such compositions to treat IgA nephropathy or Henoch-Schönlein purpura nephritis in a subject in need thereof. In certain of these embodiments, one of the two or more BAFF inhibitors is blisibimod.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Interim Futility Analyses

The BRIGHT-SC study was designed to evaluate the effects of blisibimod administration in 57 subjects with biopsy-proven IgA nephropathy. Non-placebo subjects were administered blisibimod at a dosage of 300 mg/week for eight weeks, followed by 200 mg/week for up to 104 weeks. All subjects were treated with an optimal dose of ACE inhibitor or ARB for at least 90 days prior to randomization, and these therapies were continued throughout the trial as background medication.

An interim futility analysis was performed by an independent, unblinded statistician once at least 20 subjects had completed at least eight weeks of treatment. The analysis evaluated several important biomarkers of renal disease, including proteinuria (measured as urinary protein:creatinine ratio), serum IgA, and estimated glomerular filtration rate. The analysis was considered to be positive if the proportion of responders in the blisibimod treatment group was not statistically significantly lower compared to the placebo group based on a two-sided analysis at an alpha level of 0.025. A “responder” was defined as a subject who exhibited all of the following: (1) 20% or greater decrease in proteinuria from baseline (average of screening and day 1) at any time point from week 8 to week 24; (2) greater than 7% decrease in IgA from baseline (day 1) to any time point from week 8 to week 24; and (3) no worsening (i.e., no >15% decrease) in estimated glomerular filtration rate (eGFR) from baseline (average of screening and day 1) to any time point from week 8 to week 24.

The futility analysis confirmed that blisibimod administered at a dosage of 200 mg/week or higher resulted in an improvement in several critical markers of disease activity in subjects with IgA nephropathy. Based on this analysis, the study was continued to completion as planned.

### Example 2: Interim Efficacy Analyses

Two additional interim efficacy analyses of the BRIGHT-SC study data were conducted by an independent, unblinded statistician to provide a more quantitative evaluation of blisibimod treatment effect. The first analysis was performed after approximately 50 subjects had completed 24 weeks of dosing, and the second was performed after the last subject had completed 48 weeks of dosing. All available laboratory data were evaluated, including measurements of the following critical markers of IgA nephropathy: (1) proteinuria (measured as urinary protein:creatinine ratio); (2) time-average proteinuria (averaged in 6-month and 3-month intervals); (3) estimated glomerular filtration; (4) serum immunoglobulins IgA, IgG, and IgM; (5) peripheral B cells detected by flow cytometry gated on side scatter and CD45 and expressed relative to concurrent lymphocyte counts (total B cells (CD20+CD19+), naïve B cells (CD20+CD19+IgD+CD27−), activated B cells (CD19+CD38+CD138+), memory B cells (CD19+CD27+), and plasma cells (CD19+CD27+CD38+ or CD19+CD138+CD38+)); and (5) complement C3 and C4.

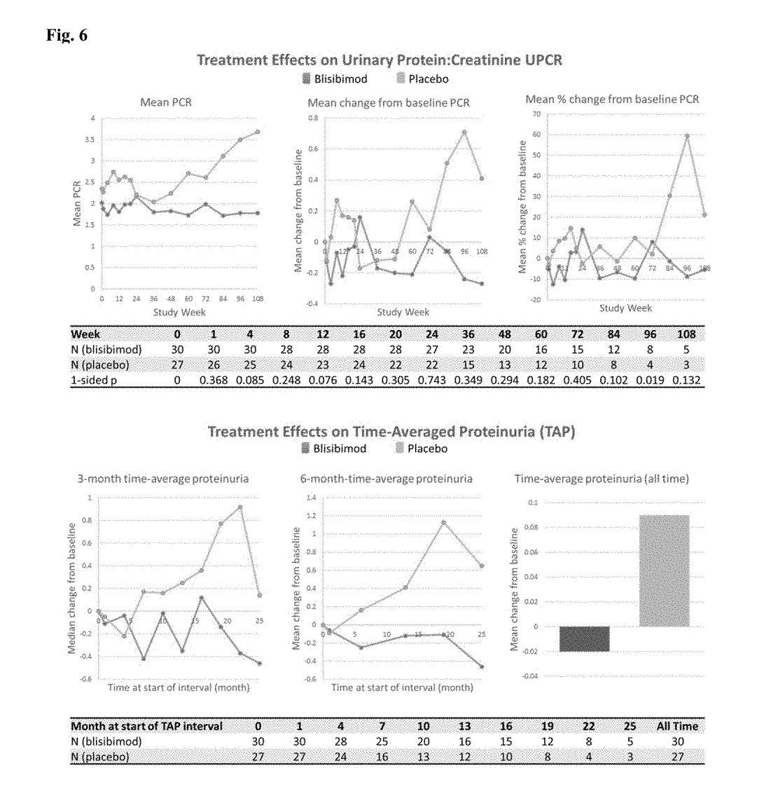

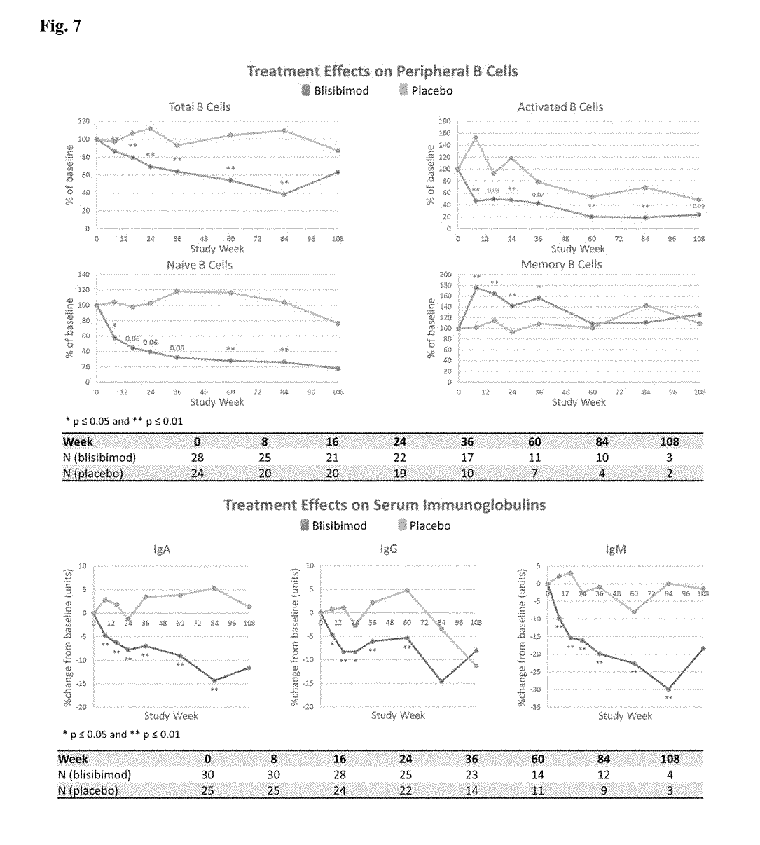

Both interim efficacy analyses showed that blisibimod administration induced clear and occasionally statistically significant treatment effects on important biomarkers of IgA renal disease after at least 24 and at least 48 weeks of treatment. In particular, blisibimod administered at a dosage of 200 mg/week or higher was associated with statistically significantly lower increases (i.e., a lower degree of worsening) in both urinary protein:creatinine ratio and time-average proteinuria (FIG. 6), and a decrease in both peripheral B cell counts and serum IgA levels (FIG. 7).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Barratt et al. J Am Soc Nephrol 27 (Abstract Edition):1167 (2016)
2. Bertsias et al. Ann Rheum Dis 71(11):1771-1782 (2012)
3. Coppo et al. Kidney Int 86(4):828-836 (2014)
4. Davin et al. Kidney Int 59(3):823-834 (2001)
5. Dooley et al. Lupus 22(1):63-72 (2013)
6. Floege Nephrol Dial Transplant 31(2):177-180 (2016a)
7. Floege & Rauen Nat Rev Nephrol 12(7):380-382 (2016b)
8. Furie et al. Arthritis Rheum 65 (Suppl 10):1742 (2013)
9. Furie et al. Ann Rheum Dis 74(9):1667-1675 (2015)
10. Hogg et al. Clin J Am Soc Nephrol 1(3):467-474 (2006)
11. Kamei et al. Pediatr Nephrol 31(5):779-785 (2016)
12. Katafuchi et al. Am J Kidney Dis 41(5):972-983 (2003)
13. Komatsu et al. Clin Exp Nephrol 20(4):552-560 (2016)
14. Koyama et al. Am J Kidney Dis 4:526-532 (1997)
15. Linnik et al. Arthritis Rheum 52(4):1129-1137 (2005)
16. Lv et al. Am J Kidney Dis 53(1):26-32 (2009)
17. McCarthy et al. J Clin Invest 121(10):3991-4002 (2011)
18. Oh et al. Kidney Int 82(12):1304-1312 (2012)
19. Petri et al. Arthritis Rheum 58(8):2453-2459 (2008)
20. Pozzi et al. Lancet 353(9156):883-887 (1999)
21. Pozzi et al. J Am Soc Nephrol 15(1):157-163 (2004)
22. Rauen et al. N Engl J Med 373(23):2225-2236 (2015)
23. Reich et al. J Am Soc Nephrol 18(12):3177-3183 (2007)
24. Stohl et al. Arthritis Rheum 64(7):2328-2337 (2012)
25. Suzuki et al. J Am Soc Nephrol 22:1795-1803 (2011)
26. Waldo Am J Kidney Dis 12(5):373-377 (1988)
27. Wyatt & Julian N Engl J Med 368(25):2402-2414 (2013)
28. Xin et al. J Nephrol 26(4):683-90 (2013)
29. Yoshikawa et al. Clin Nephrol 27(5):233-237 (1987)

1. A method of treating IgA nephropathy in a subject in need thereof comprising administering to the subject a therapeutically effective amount of a BAFF inhibitor.

2. The method of claim 1, wherein said BAFF inhibitor is blisibimod.

3. The method of claim 2, wherein blisibimod is monomeric.

4. (canceled)

5. (canceled)

6. The method of claim 2, wherein blisibimod is homodimeric or heterodimeric.

7. (canceled)

8. (canceled)

9. The method of claim 1, further comprising administering one or more second therapeutic agents selected from the group consisting of angiotensin-converting enzyme (ACE) inhibitors, angiotensin II receptor blockers (ARBs), B-cell targeted therapeutic agents, and corticosteroids.

10. The method of claim 1, wherein said BAFF inhibitor is administered as part of a composition further comprising one or more pharmaceutically acceptable excipients.

11. (canceled)

12. The method of claim 2, wherein blisibimod is administered at a dosage selected from the group consisting of 50 mg, 75 mg, 100 mg, 150 mg, 200 mg, 250 mg, and more than 250 mg per administration.

13. The method of claim 2, wherein blisibimod is administered at a dosage selected from the group consisting of 0.3 mg/kg, 1 mg/kg, 2 mg/kg, 6 mg/kg, 10 mg/kg, and more than 10 mg/kg per administration.

14. A composition comprising a BAFF inhibitor for use in the treatment of IgA nephropathy or Henoch-Schönlein purpura nephritis.

15. The composition of claim 14, wherein said BAFF inhibitor is blisibimod.

16. The composition of claim 15, wherein blisibimod is monomeric.

17. (canceled)

18. (canceled)

19. The composition of claim 15, wherein blisibimod is homodimeric or heterodimeric.

20. (canceled)

21. (canceled)

22. The composition of claim 14, further comprising one or more second therapeutic agents selected from the group consisting of angiotensin-converting enzyme (ACE) inhibitors, angiotensin II receptor blockers (ARBs), B-cell targeted therapeutic agents, and corticosteroids.

23. A kit comprising the composition of claim 14.

24. (canceled)

25. A method of treating Henoch-Schönlein purpura nephritis in a subject in need thereof comprising administering to the subject a therapeutically effective amount of a BAFF inhibitor.

26. The method of claim 25, wherein said BAFF inhibitor is blisibimod.

27. The method of claim 26, wherein blisibimod is monomeric.

28. (canceled)

29. (canceled)

30. The method of claim 26, wherein blisibimod is homodimeric or heterodimeric.

31. (canceled)

32. (canceled)

33. The method of claim 25, further comprising administering one or more second therapeutic agents selected from the group consisting of angiotensin-converting enzyme (ACE) inhibitors, angiotensin II receptor blockers (ARBs), B-cell targeted therapeutic agents, and corticosteroids.

34. The method of claim 25, wherein said BAFF inhibitor is administered as part of a composition further comprising one or more pharmaceutically acceptable excipients.

35. (canceled)

36. The method of claim 26, wherein blisibimod is administered at a dosage selected from the group consisting of 50 mg, 75 mg, 100 mg, 150 mg, 200 mg, 250 mg, and more than 250 mg per administration.

37. The method of claim 26, wherein blisibimod is administered at a dosage selected from the group consisting of 0.3 mg/kg, 1 mg/kg, 2 mg/kg, 6 mg/kg, 10 mg/kg, and more than 10 mg/kg per administration.

38-48. (canceled)


SEQUENCE LISTING (1 sequence)

SEQ ID NO: 1 - protein, 291 aa, Homo sapiens
MGCKWDLLIKQWVCDPLGSGSATGGSGSTASSGSGSATHMLPGCKWDLLIKQWVCDPLGGGGGVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK